(19) United States
(12) Patent Application Publication
(10) Pub. No.: US 2011/0223213 A1
(43) Pub. Date: Sep. 15, 2011

(54) HIGHLY PURE RANOLAZINE OR A PHARMACEUTICALLY ACCEPTABLE SALT THEREOF

(75) Inventors: Venkata Subbarao Athukuri, Andhra Pradesh (IN); Saidugari Swamy, Andhra Pradesh (IN); Chandraprakash Narani, Andhra Pradesh (IN); Mayur Devjibhai Khunt, Gujarat (IN); Nitin Sharadchandra Pradhan, Maharashtra (IN)

(73) Assignee: ACTAVIS GROUP PTC EHF, Hafnarfjorour (IS)

(21) Appl. No.: 13/124,479

(22) PCT Filed: Oct. 13, 2009
(86) PCT No.: PCT/IB09/07370

§ 371 Date: May 31, 2011

(30) Foreign Application Priority Data
Oct. 15, 2008 (IN) 2516/CHE/2008
Dec. 19, 2008 (IN) 3200/CHE/2008

(51) Int. Cl. A61K 9/14; C07D 295/096; C07D 295/15; A61K 31/495; A61P 9/00; A61P 9/10; A61P 9/06

(52) U.S. Cl. 424/400; 544/401; 544/400; 514/252.12

(57) ABSTRACT

Provided herein is an impurity of ranolazine, 1-[4-[2-hydroxy-3-(2-methoxy-phenoxy)-propyl]-piperazin-1-yl]-3-(2-methoxy-phenoxy)-propan-2-ol (dimer impurity-3), and process for preparing and isolating thereof. Provided further herein is a highly pure ranolazine or a pharmaceutically acceptable salt thereof substantially free of dimer impurity-3, process for the preparation, and pharmaceutical compositions comprising highly pure ranolazine or a pharmaceutically acceptable salt thereof substantially free of dimer impurity-3.

### CROSS REFERENCE TO RELATED APPLICATION

This application claims the benefit of priority to Indian provisional application Nos. 2516/CHE/2008, filed on Oct. 15, 2008; and 3200/CHE/2008, filed on Dec. 19, 2008; which are incorporated herein by reference in their entirety.

### FIELD OF THE DISCLOSURE

Disclosed herein are impurities of ranolazine or a pharmaceutically acceptable salt thereof, and processes for the preparation and isolation thereof. Disclosed further herein is a highly pure ranolazine or a pharmaceutically acceptable salt thereof substantially free of impurities, a process for the preparation thereof, and pharmaceutical compositions comprising highly pure ranolazine or a pharmaceutically acceptable salt thereof substantially free of impurities.

### BACKGROUND

Ranolazine, 1-[3-(2-methoxyphenoxy-2-hydroxypropyl]-4-[(2,6-dimethylphenyl)amino carbonylmethyl]piperazine, is an important antianginal and anti-ischemic agent and useful in the treatment of cardiovascular diseases, including arrhythmias, variant and exercise induced angina and myocardial infarction. Ranolazine is represented by the following structural formula I:

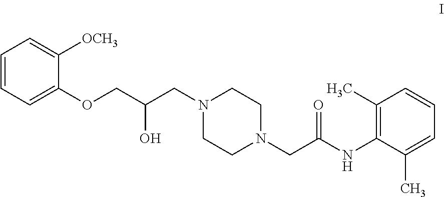

Ranolazine was approved under the brand name RANEXA®, by the U.S. Food and Drug Administration. Ranolazine was first disclosed in U.S. Pat. No. 4,567,264.

Processes for the preparation of ranolazine, related compounds, and their pharmaceutically acceptable salts were disclosed in U.S. Pat. No. 4,567,264, European Patent No. 0483932 and PCT Publication Nos. WO 2006/008753 and WO 2008/047388.

U.S. Pat. No. 4,567,264 (hereinafter referred to as the '264 patent) describes the preparation of ranolazine base by condensing 1-(2-methoxyphenoxy)-2,3-epoxypropane with 4-[(2,6-dimethylphenyl)aminocarbonylmethyl]piperazine in a solvent medium containing methanol and toluene at reflux temperature. The resulting mass was purified by column chromatography over silica gel using methanol/methylene chloride as an eluent, and the ranolazine base was isolated as an oil. The hydrochloride salt was prepared in methanol using hydrochloric acid, and the salt was isolated by addition of ether.

European Patent No. 0483932 (hereinafter referred to as the '932 patent) describes the preparation of ranolazine base by condensation of α-[N,N-bis(2-chloroethyl)amino]-2,6-dimethylacetanilide hydrochloride with 1-[3-(2-methoxyphenoxy)-2-hydroxy]-propylamine. The base was purified using column chromatography, and hydrochloride salt was formed by treating with metholic hydrochloric acid. Crystallization by addition of diethyl ether as a co-solvent yielded a product with melting point 229-230° C.

PCT Publication No. WO 2006/008753 A1 (hereinafter referred to as the '753 application) discloses two polymorphic forms (Form A and amorphous form) of ranolazine dihydrochloride and a crystalline form of ranolazine base, and characterizes them by powder X-ray diffraction (P-XRD) and Differential Scanning Calorimetry (DSC). According to the '753 application, the ranolazine base is isolated as crystalline form by neutralizing the ranolazine dihydrochloride with liquor ammonia in a solvent mixture containing water and acetone, followed by precipitation.

PCT Publication No. WO 2008/047388 A1 (hereinafter referred to as the '388 application) describes a process for the preparation of ranolazine and pharmaceutically acceptable salts thereof, by reacting 2,6-dimethylaniline derivative with chloroacetyl chloride in the presence of base in water. The resulting amide intermediate is reacted with piperazine, and the resulting piperazine derivative is further condensed with an appropriate oxirane derivative (prepared by the reaction of 2-methoxyphenol with epichlorohydrin in the presence of base using phase transfer catalyst) in an inert solvent to produce crude ranolazine. The crude ranolazine is further purified by crystallizing ranolazine base from an organic solvent selected from alcohols or aromatic hydrocarbons.

Two impurities of ranolazine have been disclosed in the '388 application. These impurities are characterized as 1,3-bis-(2-methoxyphenoxy)-propan-2-ol (hereinafter referred to as the ‘Dimer Impurity-1’) and N-(2,6-dimethyl-phenyl)-2-[4-[(2,6-dimethyl-phenylcarbamoyl)-methyl]-piperazin-1-yl]-acetamide (hereinafter referred to as the ‘Dimer Impurity-2’), and which have the following structural formulae:

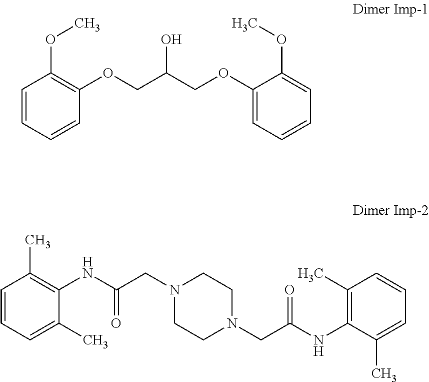

Ranolazine obtained by the processes described in the above prior art does not have satisfactory purity for pharmaceutical use. Unacceptable amounts of impurities are generally formed along with ranolazine. In addition, the processes involve the additional step of column chromatographic purifications. Methods involving column chromatographic purifications are generally undesirable for large-scale operations as they require additional expensive setup adding to the cost of production, thereby making the processes commercially unfeasible.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in ranolazine or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure ranolazine or a pharmaceutically acceptable salt thereof substantially free of impurities, as well as processes for preparing thereof.

### SUMMARY

In one aspect, provided herein is a dimer compound, 1-[4-[2-hydroxy-3-(2-methoxy-phenoxy)-propyl]-piperazin-1-yl]-3-(2-methoxy-phenoxy)-propan-2-ol, having the following structural formula II:

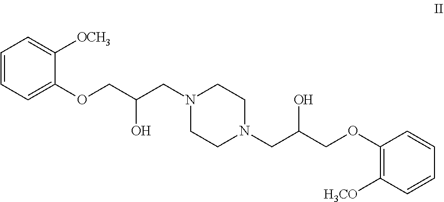

In another aspect, provided herein is an impurity of ranolazine, dimer impurity-3, 1-[4-[2-hydroxy-3-(2-methoxy-phenoxy)-propyl]-piperazin-1-yl]-3-(2-methoxy-phenoxy)-propan-2-ol, of formula II.

In another aspect, encompassed herein is a process for synthesizing and isolating the dimer compound of formula II, also referred to as the “dimer impurity-3”.

In another aspect, provided herein is a highly pure ranolazine or a pharmaceutically acceptable salt thereof substantially free of dimer impurity-3.

In another aspect, provided herein is a highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities.

In another aspect, provided herein is a highly pure ranolazine or a pharmaceutically acceptable salt thereof substantially free of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

In another aspect, encompassed herein is a process for preparing the highly pure ranolazine or a pharmaceutically acceptable salt thereof substantially free of dimer impurity-3.

In yet another aspect, encompassed herein is a process for preparing the highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities.

In still further aspect, encompassed herein is a process for preparing the highly pure ranolazine or a pharmaceutically acceptable salt thereof substantially free of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

In another aspect, provided herein is a pharmaceutical composition comprising highly pure ranolazine or a pharmaceutically acceptable salt thereof substantially free of dimer impurity-3, and one or more pharmaceutically acceptable excipients.

In still another aspect, provided herein is a pharmaceutical composition comprising highly pure ranolazine or a pharmaceutically acceptable salt thereof substantially free of dimer impurity-3 made by the process disclosed herein, and one or more pharmaceutically acceptable excipients.

In still further aspect, encompassed is a process for preparing a pharmaceutical formulation comprising combining highly pure ranolazine or a pharmaceutically acceptable salt thereof substantially free of dimer impurity-3 with one or more pharmaceutically acceptable excipients.

In another aspect, the highly pure ranolazine or a pharmaceutically acceptable salt thereof substantially free of dimer impurity-3 disclosed herein for use in the pharmaceutical compositions has a 90 volume-percent of the particles (D₉₀) of less than or equal to about 400 microns, specifically less than or equal to about 300 microns, more specifically less than or equal to about 100 microns, still more specifically less than or equal to about 60 microns, and most specifically less than or equal to about 15 microns.

### DETAILED DESCRIPTION

According to one aspect, there is provided a ranolazine dimer compound, 1-[4-[2-hydroxy-3-(2-methoxy-phenoxy)-propyl]-piperazin-1-yl]-3-(2-methoxy-phenoxy)-propan-2-ol, having the following structural formula II:

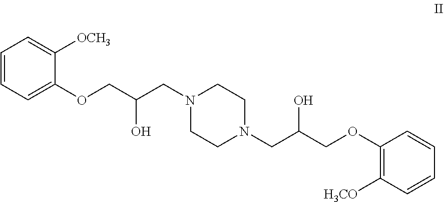

According to another aspect, there is provided an impurity of ranolazine, ranolazine dimer impurity-3, 1-[4-[2-hydroxy-3-(2-methoxy-phenoxy)-propyl]-piperazin-1-yl]-3-(2-methoxy-phenoxy)-propan-2-ol, of formula II.

The ranolazine dimer impurity-3 has been identified, isolated and synthesized. The dimer impurity-3 was detected and resolved from ranolazine by HPLC with an RRt of 0.86. The structure of the compound of formula II was deduced with the aid of ¹H, ¹³C NMR and IR spectroscopy and FAB mass spectrometry. The parent ion at 446.5 is consistent with the assigned structure.

According to another aspect, there is provided an isolated ranolazine dimer impurity-3.

In one embodiment, the dimer compound of formula II is prepared as per the process exemplified in the Example 23 as disclosed herein.

It has been found that the following three unreacted process intermediates (Impurity-4, Impurity-5 and Impurity-6) are also formed as impurities in the synthesis of ranolazine and remain in the final product:
i) Impurity-4: 2-[(2-methoxyphenoxy)methyl]oxirane, which has the following structural formula:

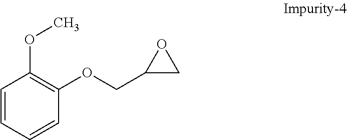

and it is detected and resolved from ranolazine by HPLC with an RRt of 0.37;
ii) Impurity-5: 2-chloro-N-(2,6-dimethylphenyl)acetamide, which has the following structural formula:

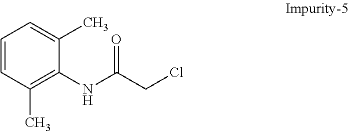

and it is detected and resolved from ranolazine by HPLC with an RRt of 0.46;
iii) Impurity-6: 4-[(2,6-dimethylphenyl)aminocarbonylmethyl]piperazine, which has the following structural formula:

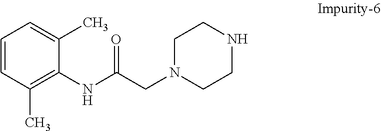

and it is detected and resolved from ranolazine by HPLC with an RRt of 0.13.

In addition to the above impurities, there are six other impurities identified at 0.58, 0.78, 1.02, 1.07, 1.16 and 1.23±0.01 RRt's (hereinafter referred to as the ‘0.58 RRt’ impurity, ‘0.78 RRt’ impurity, ‘1.02 RRt’ impurity, ‘1.07 RRt’ impurity, ‘1.16 RRt’ impurity and ‘1.23 RRt’ impurity, collectively referred to as the ‘single maximum unknown impurities’), whose presence was observed in ranolazine.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.01.

Thus there is a need for a method for determining the level of impurities in ranolazine samples and removing the impurities.

Extensive experimentation was carried out by the present inventors to reduce the level of the dimer impurity-3, ‘0.58 RRt’, ‘0.78 RRt’, ‘1.02 RRt’, ‘1.07 RRt’, ‘1.16 RRt’ and ‘1.23 RRt’ impurities in ranolazine. As a result, it has been found that the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities formed in the preparation of the ranolazine can be reduced or completely removed by the purification process disclosed herein.

According to another aspect, there is provided a highly pure ranolazine or a pharmaceutically acceptable salt thereof substantially free of dimer impurity-3.

In one embodiment, the highly pure ranolazine or a pharmaceutically acceptable salt thereof disclosed herein is substantially free from at least one, or more, specifically all, of the dimer impurity-3, ‘0.58 RRt’, ‘0.78 RRt’, ‘1.02 RRt’, ‘1.07 RRt’, ‘1.16 RRt’ and ‘1.23 RRt’ impurities.

In another embodiment, the highly pure ranolazine or a pharmaceutically acceptable salt thereof disclosed herein is substantially free from at least one, or more, specifically all, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

According to another aspect, there is provided a highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities.

According to another aspect, there is provided a highly pure ranolazine or a pharmaceutically acceptable salt thereof substantially free of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

As used herein, “highly pure ranolazine or a pharmaceutically acceptable salt thereof substantially free of dimer impurity-3” refers to ranolazine or a pharmaceutically acceptable salt thereof comprising the dimer impurity-3 in an amount of less than about 0.15 area-% as measured by HPLC. Specifically, the ranolazine, as disclosed herein, contains less than about 0.1 area-%, more specifically less than about 0.05 area-%, still more specifically less than about 0.02 area-% of the dimer impurity-3, and most specifically is essentially free of the dimer impurity-3.

In one embodiment, the highly pure ranolazine or a pharmaceutically acceptable salt thereof disclosed herein comprises the dimer impurity-3 in an amount of about 0.01 area-% to about 0.15 area-%, specifically in an amount of about 0.01 area-% to about 0.05 area-%, as measured by HPLC.

As used herein, “highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities” refers to ranolazine or a pharmaceutically acceptable salt thereof comprising one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities, each one, in an amount of less than about 0.15 area-% as measured by HPLC. Specifically, the ranolazine, as disclosed herein, contains less than about 0.1 area-%, more specifically less than about 0.05 area-%, still more specifically less than about 0.02 area-% of one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities, and most specifically is essentially free of one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities.

In one embodiment, the highly pure ranolazine or a pharmaceutically acceptable salt thereof disclosed herein comprises one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities, each one, in an amount of about 0.01 area-% to about 0.15 area-%, specifically in an amount of about 0.01 area-% to about 0.05 area-%, as measured by HPLC.

As used herein, “highly pure ranolazine or a pharmaceutically acceptable salt thereof substantially free of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities” refers to ranolazine or a pharmaceutically acceptable salt thereof comprising one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities, each one, in an amount of less than about 0.15 area-% as measured by HPLC. Specifically, the ranolazine, as disclosed herein, contains less than about 0.1 area-%, more specifically less than about 0.05 area-%, still more specifically less than about 0.02 area-% of one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities, and most specifically is essentially free of one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

In another embodiment, the highly pure ranolazine or a pharmaceutically acceptable salt thereof disclosed herein has a total purity of greater than about 99%, specifically greater than about 99.5%, more specifically greater than about 99.9%, and most specifically greater than about 99.95% as measured by HPLC. For example, the purity of the highly pure ranolazine or a pharmaceutically acceptable salt thereof is about 99% to about 99.9%, or about 99.5% to about 99.99%.

In another embodiment, the highly pure ranolazine or a pharmaceutically acceptable salt thereof disclosed herein is essentially free of one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

In yet another embodiment, the highly pure ranolazine or a pharmaceutically acceptable salt thereof disclosed herein is essentially free of the dimer impurity-1 and dimer impurity-2.

The term “ranolazine or a pharmaceutically acceptable salt thereof essentially free of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities” refers to ranolazine or a pharmaceutically acceptable salt thereof contains a non-detectable amount of one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities as measured by HPLC.

The term “ranolazine or a pharmaceutically acceptable salt thereof essentially free of dimer impurity-1 and dimer impurity-2” refers to ranolazine or a pharmaceutically acceptable salt thereof contains a non-detectable amount of the dimer impurity-1 and dimer impurity-2.

According to another aspect, there is provided a process for preparing highly pure ranolazine or a pharmaceutically acceptable salt thereof substantially free of one, or more, specifically all, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities, comprising:
a) providing a first solution of crude ranolazine in a first solvent or a solvent medium comprising a first solvent and a second solvent, wherein the first solvent is selected from the group consisting of a polar aprotic solvent, a ketone, and mixtures thereof, and wherein the second solvent is selected from the group consisting of water, an alcohol, an ester, and mixtures thereof;
b) optionally, subjecting the first solution to carbon treatment or silica gel treatment;
c) optionally, combining the first solution obtained in step-(a) or step-(b) with the second solvent to produce a second solution or suspension;
d) isolating and/or recovering highly pure ranolazine substantially free of the impurities either from the first solution obtained in step-(a) or step-(b), or from the second solution or suspension obtained in step-(c); and
e) optionally, converting the highly pure ranolazine obtained in step-(d) into a pharmaceutically acceptable salt thereof.

As per the stability studies have been conducted by the present inventors, the highly pure ranolazine substantially free of the impurities obtained by the process disclosed herein is found to be more stable.

In one embodiment, the highly pure ranolazine substantially free of the impurities obtained by the process disclosed herein remains stable, when stored at a temperature of about 25±2° C. and at a relative humidity of about 60±5% for a period of 6 months.

In another embodiment, the highly pure ranolazine substantially free of the impurities obtained by the process disclosed herein remains stable, when stored at a temperature of about 40±2° C. and at a relative humidity of about 75±5% for a period of 6 months.

The term “remains stable”, as defined herein, refers to lack of formation of impurities, while being stored as described hereinbefore.

Exemplary first solvents used in step-(a) include, but are not limited to, acetone, methyl ethyl ketone, methyl isobutyl ketone, methyl tert-butyl ketone, N,N-dimethylformamide, N,N-dimethylacetamide, dimethylsulfoxide, tetrahydrofuran, and mixtures thereof. The term solvent also includes mixtures of solvents.

In one embodiment, the first solvent is selected from the group consisting of acetone, N,N-dimethylformamide, N,N-dimethylacetamide, dimethylsulfoxide, tetrahydrofuran, and mixtures thereof. Specifically, the first solvent is selected from the group consisting of N,N-dimethylacetamide, dimethylsulfoxide, tetrahydrofuran, and mixtures thereof.

Exemplary second solvents used in step-(a) or step-(c) include, but are not limited to, water, methanol, ethanol, n-propanol, isopropyl alcohol, isobutanol, n-butanol, tert-butanol, amyl alcohol, isoamyl alcohol, ethyl acetate, methyl acetate, isopropyl acetate, tert-butyl methyl acetate, ethyl formate, and mixtures thereof.

In one embodiment, the second solvent is selected from the group consisting of water, methanol, ethanol, n-propanol, isopropyl alcohol, isobutanol, n-butanol, tert-butanol, ethyl acetate, and mixtures thereof. Specifically, the second solvent is selected from the group consisting of water, methanol, ethanol, isopropyl alcohol, ethyl acetate, and mixtures thereof.

Step-(a) of providing a solution of crude ranolazine includes dissolving crude ranolazine in the first solvent or the solvent medium comprising the first and second solvents, or obtaining an existing solution from a previous processing step.

In one embodiment, the crude ranolazine is dissolved in the first solvent or in the solvent medium at a temperature of above about 25° C., specifically at about 25° C. to about 110° C., and more specifically at about 40° C. to about 80° C.

In another embodiment, the first solution of crude ranolazine in the solvent medium comprising the first and second solvents in step-(a) is provided either by dissolving crude ranolazine in a mixture of the first and second solvents or by dissolving crude ranolazine in the first solvent at a temperature of above about 25° C. to form a solution and followed by combining the solution with the second solvent at the same temperature to form the first solution.

In another embodiment, the first solution in step-(a) is prepared by reacting 1-(2-methoxyphenoxy)-2,3-epoxypropane with 1-[(2,6-dimethylphenyl)aminocarbonylmethyl]piperazine in a reaction inert solvent under suitable conditions to produce a reaction mass containing ranolazine free base, followed by usual work up such as washings, extractions, evaporations, filtrations, pH adjustments, or a combination thereof. In one embodiment, the work-up includes dissolving or extracting the resulting ranolazine in the first solvent or in the solvent medium at a temperature of above about 25° C., specifically at about 25° C. to about 110° C., and more specifically at about 40° C. to about 80° C.

Alternatively, the first solution in step-(a) is prepared by treating an acid addition salt of ranolazine with a base to produce ranolazine free base followed by extracting or dissolving the ranolazine in the first solvent or in the solvent medium at a temperature of above about 25° C., specifically at about 25° C. to about 110° C., and more specifically at about 40° C. to about 80° C.

In another embodiment, the acid addition salt of ranolazine is derived from a therapeutically acceptable acid such as hydrochloric acid, acetic acid, propionic acid, sulfuric acid, nitric acid, succinic acid, maleic acid, fumaric acid, citric acid, glutaric acid, citraconic acid, glutaconic acid, tartaric acid, malic acid, and ascorbic acid. A specific salt is ranolazine dihydrochloride.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

In one embodiment, the base is an organic or inorganic base. Specific organic bases are triethyl amine, trimethylamine and N,N-diisopropylethylamine.

In another embodiment, the base is an inorganic base. Exemplary inorganic bases include, but are not limited to, aqueous ammonia; hydroxides, alkoxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

The first solution obtained in step-(a) is optionally stirred at a temperature of about 25° C. to about 110° C. for at least 15 minutes and specifically at a temperature of about 40° C. to about 80° C. for about 20 minutes to about 8 hours.

The carbon treatment or silica gel treatment in step-(b) is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing ranolazine free base by removing charcoal or silica gel. Specifically, the finely powdered carbon is an active carbon. A specific mesh size of silica gel is 40-500 mesh, and more specifically 60-120 mesh.

Combining of the first solution with the second solvent in step-(c) is done in a suitable order, for example, the first solution is added to the second solvent, or alternatively, the second solvent is added to the first solution. The addition is, for example, carried out drop wise or in one portion or in more than one portion. The addition is specifically carried out at a temperature of above about 25° C., more specifically at about 30° C. to about 110° C., and most specifically at about 40° C. to about 80° C. under stirring. After completion of the addition process, the resulting mass is stirred at a temperature of above about 25° C. for at least 10 minutes, specifically at about 30° C. to about 110° C. for about 20 minutes to about 10 hours, and more specifically at a temperature of about 40° C. to about 80° C. for about 30 minutes to about 4 hours to produce a second solution or suspension.

The isolation of highly pure ranolazine base in step-(d) is carried out, for example, by forcible or spontaneous crystallization.

Spontaneous crystallization refers to crystallization without the help of an external aid, such as seeding, cooling etc., and forcible crystallization refers to crystallization with the help of an external aid.

Forcible crystallization is initiated by methods such as cooling, seeding, partial removal of the solvent from the solution, by combining an anti-solvent with the solution, or a combination thereof.

In one embodiment, the crystallization is carried out by cooling the solution while stirring at a temperature of below 35° C. for at least 15 minutes, specifically at about 0° C. to about 30° C. for about 30 minutes to about 20 hours, and more specifically at about 20° C. to about 30° C. for about 2 hours to about 10 hours.

The recovery of highly pure ranolazine base in step-(d) is accomplished by techniques such as filtration, filtration under vacuum, decantation, centrifugation, or a combination thereof. In one embodiment, the ranolazine base is recovered by filtration employing a filtration media of, for example, a silica gel or celite.

Pharmaceutically acceptable salts of ranolazine in step-(e) can be prepared in high purity by using the highly pure ranolazine base substantially free of impurities obtained by the method disclosed herein, by known methods, for example as described in U.S. Pat. No. 4,567,264.

Specific pharmaceutically acceptable salts of ranolazine include, but are not limited to, dihydrochloride, dihydrobromide, oxalate, maleate, fumarate, besylate, tosylate, tartrate, and more specifically dihydrochloride salt.

The highly pure ranolazine or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a highly pure ranolazine or a pharmaceutically acceptable salt thereof substantially free of at least one, or more, specifically all, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities, has a relatively low content of one or more organic volatile impurities.

In one embodiment, the ranolazine or a pharmaceutically acceptable salt thereof obtained by the purification process disclosed herein comprises less than about 3000 parts per million (ppm) methanol, less than about 5000 ppm acetone, less than about 300 ppm methylene chloride, less than about 5000 ppm methyl ethyl ketone, less than about 5000 ppm ethyl acetate, less than about 3000 ppm cyclohexane, and less than about 1000 ppm N,N-dimethylacetamide, as measured by Gas Chromatography (GC). Specifically, the ranolazine obtained by the purification process disclosed herein comprises less than about 300 parts per million (ppm) methanol, less than about 1000 ppm acetone, less than about 50 ppm methylene chloride, less than about 500 ppm methyl ethyl ketone, less than about 500 ppm ethyl acetate, less than about 300 ppm cyclohexane, and less than about 7000 ppm N,N-dimethylacetamide.

In another embodiment, ranolazine or a pharmaceutically acceptable salt thereof substantially free of impurities obtained by the purification process disclosed herein has the overall level of organic volatile impurities less than about 1500 ppm, specifically less than about 1000 ppm, and most specifically less than about 500 ppm.

Further encompassed herein is the use of the highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities for the manufacture of a pharmaceutical composition together with a pharmaceutically acceptable carrier.

A specific pharmaceutical composition of highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities is selected from a solid dosage form and an oral suspension.

In one embodiment, the highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from cardiovascular diseases including arrhythmias, variant and exercise induced angina and myocardial infarction, comprising administering a therapeutically effective amount of the highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities, or a pharmaceutical composition that comprises a therapeutically effective amount of highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities, along with pharmaceutically acceptable excipients.

According to another aspect, there is provided pharmaceutical compositions comprising highly pure ranolazine or a pharmaceutically acceptable salt thereof substantially free of dimer impurity-3 prepared according to the processes disclosed herein and one or more pharmaceutically acceptable excipients.

According to another aspect, there is provided a process for preparing a pharmaceutical formulation comprising combining highly pure ranolazine or a pharmaceutically acceptable salt thereof substantially free of dimer impurity-3 prepared according to processes disclosed herein, with one or more pharmaceutically acceptable excipients.

According to another aspect, there is provided pharmaceutical compositions comprising highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities prepared according to the processes disclosed herein and one or more pharmaceutically acceptable excipients.

Yet in another embodiment, pharmaceutical compositions comprise at least a therapeutically effective amount of highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure ranolazine or a pharmaceutically acceptable salt thereof substantially free of at least one, or more, of the dimer impurity-3, ‘0.58 RRt’, ‘0.78 RRt’, ‘1.02 RRt’, ‘1.07 RRt’, ‘1.16 RRt’ and ‘1.23 RRt’ impurities within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Experimental

### High Performance Liquid Chromatography (HPLC):

The purity was measured by High Performance Liquid Chromatography by using Waters, alliance 2695 HPLC system having dual wavelength UV detector with Empower chromatography software or its equivalent under the following conditions:
Column: Altima HP C18 (150×4.6 mm, 3.4 μm), Make: Alltech Part No. 87688,
Column oven temperature: 40° C.
Detection: UV at 215 nm
Flow rate: 1.0 mL/minute
Injection volume: 10 μL
Run time: 70 minutes
Diluent: Methanol

The following examples are given for the purpose of illustrating the present disclosure and should not be considered as limitation on the scope or spirit of the disclosure.

### EXAMPLES

### Example 1

Preparation of Crude Ranolazine

A mixture of 1-[(2,6-Dimethylphenyl)aminocarbonylmethyl]piperazine (125 gm), 1-(2-methoxy phenoxy)-2,3-epoxypropane (96 gm) and methanol (750 ml) was refluxed for 6-8 hours at 65° C. The reaction mixture was initially cooled to 25-30° C., followed by cooling to 0-5° C. and stirring for 4 hours. The resulting solid was filtered, the product was washed with pre cooled methanol (60 ml), and the material was suction dried followed by drying under vacuum at 50-55° C. to give 175 gm of crude ranolazine.

Content of Impurities: ‘0.58 RRt’ impurity: 0.06%; ‘1.16 RRt’ impurity: 0.06%; dimer impurity-1: 0.16%; dimer impurity-2: 0.05%; dimer impurity-3: 0.22%; impurity-4: 0.08%; impurity-5: 0.01%; and impurity-6: 0.07%.

### Example 2

Purification of Crude Ranolazine

Crude ranolazine (10 gm, Content of Impurities: ‘0.78 RRt’ impurity: 0.28%; ‘1.23 RRt’ impurity: 0.05%; dimer impurity-1: 0.11%; dimer impurity-2: 0.02%; dimer impurity-3: 0.17%; HPLC Purity: 98.72%) was dissolved in tetrahydrofuran (38 ml) at 45-55° C. The solution was filtered through a hyflo bed and washed with tetrahydrofuran (5 ml) at 45-55° C. The resulting solution was followed by slow addition of water (170 ml) at the same temperature. The reaction mixture was cooled to 0-5° C. and stirred for 4 hours. The resulting product was filtered and washed with chilled water (20 ml). The resulting solid was dried under vacuum at 50-55° C. to give 9 gm of pure ranolazine (HPLC Purity: 99.96%).

Content of Impurities: ‘0.78 RRt’ impurity: Below detection limit (BDL); ‘1.23 RRt’ impurity: Below detection limit; Dimer Impurity-1: Below detection limit; Dimer Impurity-2: Below detection limit; Dimer Impurity-3: Below detection limit.

### Example 3

Purification of Crude Ranolazine

Crude ranolazine (10 gm, Content of Impurities: ‘1.02 RRt’ impurity: 0.40%; ‘1.16 RRt’ impurity: 0.07%; dimer impurity-1: 0.06%; dimer impurity-2: 0.04%; dimer impurity-3: 0.08%; HPLC Purity: 98.70%) was dissolved in tetrahydrofuran (38 ml) at 45-55° C. The solution was filtered through a hyflo bed and washed with tetrahydrofuran (5 ml) at 45-55° C. The resulting solution was followed by slow addition of water (170 ml) at the same temperature. The reaction mixture was cooled to 0-5° C. and stirred for 4 hours. The resulting product was filtered and washed with chilled water (20 ml). The resulting solid was dried under vacuum at 50-55° C. to give 9 gm of pure ranolazine (HPLC Purity: 99.96%).

Content of Impurities: ‘1.02 RRt’ impurity: Below detection limit; ‘1.16 RRt’ impurity: 0.05%; Dimer Impurity-1: Below detection limit; Dimer Impurity-2: Below detection limit; Dimer Impurity-3: Below detection limit

### Example 4

Purification of Crude Ranolazine

Crude ranolazine (10 gm, Content of Impurities: ‘0.58 RRt’ impurity: 0.28%; ‘1.16 RRt’ impurity: 0.09%; dimer impurity-1: 0.11%; dimer impurity-2: 0.02%; dimer impurity-3: 0.17%; HPLC Purity: 98.72%) was dissolved in tetrahydrofuran (38 ml) at 45-55° C. The solution was filtered through a hyflo bed and washed with tetrahydrofuran (5 ml) at 45-55° C. The resulting solution was followed by slow addition of water (170 ml) at the same temperature. The reaction mixture was cooled to 0-5° C. and stirred for 4 hours. The resulting product was filtered and washed with chilled water (20 ml). The resulting solid was dried under vacuum at 50-55° C. to give 9 gm of pure ranolazine (HPLC Purity: 99.94%).

Content of Impurities: ‘0.58 RRt’ impurity: 0.01%; ‘1.16 RRt’ impurity: 0.04%; Dimer Impurity-1: Below detection limit; Dimer Impurity-2: Below detection limit; Dimer Impurity-3: Below detection limit.

### Example 5

Purification of Crude Ranolazine

Crude ranolazine (10 gm, Content of Impurities: ‘0.78 RRt’ impurity: 0.40%; ‘1.23 RRt’ impurity: 0.09%; dimer impurity-1: 0.06%; dimer impurity-2: 0.04%; dimer impurity-3: 0.08%; HPLC Purity: 98.70%) was dissolved in tetrahydrofuran (70 ml) at 45-55° C. The solution was filtered through a hyflo bed and washed with tetrahydrofuran (7 ml) at 45-55° C. The reaction mixture was cooled to 0-5° C. and stirred for 4 hours. The resulting product was filtered and washed with chilled tetrahydrofuran. The resulting solid was dried under vacuum at 50-55° C. to give 7.3 gm of pure ranolazine (HPLC Purity: 99.90%).

Content of Impurities: ‘0.78 RRt’ impurity: Below detection limit; ‘1.23 RRt’ impurity: Below detection limit; Dimer Impurity-1: Below detection limit; Dimer Impurity-2: Below detection limit; Dimer Impurity-3: Below detection limit.

### Example 6

Purification of Crude Ranolazine

Crude ranolazine (10 gm, Content of Impurities: ‘0.78 RRt’ impurity: 0.36%; dimer impurity-1: 0.27%; HPLC Purity: 98.40%) was dissolved in tetrahydrofuran (70 ml) at 45-55° C. The solution was filtered through a hyflo bed and washed with tetrahydrofuran (7 ml) at 45-55° C. The reaction mixture was cooled to 0-5° C. and stirred for 4 hours. The resulting product was filtered and washed with chilled tetrahydrofuran. The resulting solid was dried under vacuum at 50-55° C. to give 7.3 gm of pure ranolazine (HPLC Purity: 99.87%).

Content of Impurities: ‘0.78 RRt’ impurity: Below detection limit; Dimer Impurity-1: Below detection limit.

### Example 7

Purification of Crude Ranolazine

Crude ranolazine (10 gm, Content of Impurities: ‘1.07 RRt’ impurity: 0.19; dimer impurity-2: 0.01%; dimer impurity-3: 0.02%; HPLC Purity: 99.66%) was dissolved in tetrahydrofuran (70 ml) at 45-55° C. The solution was filtered through a hyflo bed and washed with tetrahydrofuran (7 ml) at 45-55° C. The reaction mixture was cooled to 0-5° C. and stirred for 4 hours. The resulting product was filtered and washed with chilled tetrahydrofuran. The resulting solid was dried under vacuum at 50-55° C. to give 7.3 gm of pure ranolazine (HPLC Purity: 99.90%).

Content of Impurities: ‘1.07 RRt’ impurity: 0.09%; Dimer Impurity-2: Below detection limit; Dimer Impurity-3: Below detection limit.

### Example 8

Purification of Crude Ranolazine

Crude ranolazine (10 gm, Content of Impurities: ‘0.78 RRt’ impurity: 0.21%; ‘1.23 RRt’ impurity: 0.31%; dimer impurity-1: 0.09%; HPLC Purity: 98.64%) was dissolved in acetone (70 ml) at 50-55° C. The solution was followed by the slow addition of water (245 ml) at 50-55° C. The reaction mixture was cooled to 25-35° C. and then stirred for 2 hours. The resulting product was filtered, washed with water and then dried under vacuum at 50-55° C. to give 8.6 gm of ranolazine (HPLC Purity: 99.73%).

Content of Impurities: ‘0.78 RRt’ impurity: Below detection limit; ‘1.23 RRt’ impurity: 0.14%; Dimer Impurity-1: Below detection limit.

### Example 9

Purification of Crude Ranolazine

Crude ranolazine (10 gm, Content of Impurities: ‘0.78 RRt’ impurity: 0.21%; ‘1.23 RRt’ impurity: 0.31%; dimer impurity-1: 0.09%; HPLC Purity: 98.64%) was dissolved in acetone (70 ml) at 50-55° C. The reaction mixture was cooled to 25-35° C. and then stirred for 2 hours. The resulting product was filtered, washed with chilled acetone and then dried under vacuum at 50-55° C. to give 7.3 gm of ranolazine (HPLC Purity: 99.58%).

Content of Impurities: ‘0.78 RRt’ impurity: Below detection limit; ‘1.23 RRt’ impurity: 0.17%;

Dimer Impurity-1: Below detection limit.

### Example 10

Purification of Crude Ranolazine

Crude ranolazine (10 gm, HPLC Purity: 98.95%) was added to N,N-dimethylacetamide (20 ml) and then heated at 50-55° C. to form a clear solution. The solution was stirred for 10 minutes at 50-55° C. followed by the addition of acetone (100 ml) over a period of 30 minutes at 50-55° C., and stirring for 10 minutes at the same temperature. The resulting solution was cooled to 25-35° C. and stirred for 6-8 hours. The resulting mass was then allowed to cool at 0-5° C. and stirred for 4 hours at the same temperature. The resulting solid was filtered, the product was washed with pre cooled acetone (10 ml) followed by suction drying and then drying at 50-55° C. under vacuum to give 7.5 gm of pure ranolazine (HPLC Purity: 99.97%).

Content of Impurities:

Before Purification: ‘0.58 RRt’ impurity: 0.06%; ‘1.16 RRt’ impurity: 0.06%; dimer impurity-1: 0.16%; dimer impurity-2: 0.05%; dimer impurity-3: 0.22%; impurity-4: 0.08%; impurity-5: 0.01%; and impurity-6: 0.07%.

After Purification: ‘0.58 RRt’ impurity: Below detection limit; ‘1.16 RRt’ impurity: 0.01%; dimer impurity-1: Below detection limit; dimer impurity-2: Below detection limit; dimer impurity-3: Below detection limit; impurity-4: Below detection limit; impurity-5: Below detection limit; and impurity-6: Below detection limit.

Level of organic volatile impurities: Acetone—345 ppm; N,N-dimethylacetamide—292 ppm.

### Example 11

Purification of Crude Ranolazine

Crude ranolazine (10 gm, HPLC Purity: 99.47%) was added to N,N-dimethylacetamide (20 ml) and then heated at 50-55° C. to form a clear solution. The solution was stirred for 10 minutes at 50-55° C. followed by the addition of acetone (100 ml) over a period of 30 minutes at 50-55° C., and then stirring for 10 minutes at the same temperature. The resulting solution was cooled to 25-35° C. and stirred for 6-8 hours. The resulting solid was filtered, the product was washed with pre cooled acetone (10 ml) followed by suction drying and then drying at 50-55° C. under vacuum to give 6.5 gm of pure ranolazine (HPLC Purity: 99.96%).

Content of Impurities:

Before Purification: ‘0.58 RRt’ impurity: 0.02%; ‘1.16 RRt’ impurity: 0.06%; dimer impurity-1: 0.05%; dimer impurity-2: 0.03%; dimer impurity-3: 0.16%; impurity-4: 0.03%; and impurity-6: 0.04%.

After Purification: ‘0.58 RRt’ impurity: Below detection limit; ‘1.16 RRt’ impurity: 0.01%; dimer impurity-1: Below detection limit; dimer impurity-2: Below detection limit; dimer impurity-3: Below detection limit; impurity-4: Below detection limit; and impurity-6: Below detection limit.

Level of organic volatile impurities: Acetone—293 ppm; N,N-dimethylacetamide—237 ppm.

### Example 12

Purification of Crude Ranolazine

Crude ranolazine (10 gm, HPLC Purity: 99.42%) was added to a mixture of N,N-dimethylacetamide (20 ml) and acetone (100 ml), the resulting mixture was heated at 50-55° C. to form a clear solution, and then the mixture was stirred for 10 minutes at 50-55° C. The resulting solution was cooled to 25-35° C. and stirred for 6-8 hours. The resulting mass was then allowed to cool at 0-5° C. and stirred for 4 hours at the same temperature. The resulting solid was filtered, the product was washed with pre cooled acetone (10 ml) followed by suction drying and then drying at 50-55° C. under vacuum to give 6.7 gm of pure ranolazine (HPLC Purity: 99.92%).

Content of Impurities:

Before Purification: ‘0.58 RRt’ impurity: 0.01%; ‘1.16 RRt’ impurity: 0.03%; dimer impurity-1: 0.05%; dimer impurity-2: 0.02%; dimer impurity-3: 0.02%; impurity-4: 0.07%; impurity-5: 0.01%; and impurity-6: 0.17%.

After Purification: ‘0.58 RRt’ impurity: Below detection limit; ‘1.16 RRt’ impurity: 0.03%; dimer impurity-1: Below detection limit; dimer impurity-2: Below detection limit; dimer impurity-3: Below detection limit; impurity-4: Below detection limit; impurity-5: Below detection limit; and impurity-6: 0.03%.

### Example 13

Purification of Crude Ranolazine

Crude ranolazine (5 gm, HPLC Purity: 99.42%) was added to acetone (45 ml), the contents were heated at 55-57° C. to form a clear solution and then stirred for 10 minutes at the same temperature. The resulting solution was cooled to 25-35° C. and stirred for 6-8 hours. The resulting mass was then allowed to cool at 0-5° C. and stirred for 4 hours at the same temperature. The resulting solid was filtered, the product was washed with pre cooled acetone (10 ml) followed by suction drying and then drying at 50-55° C. under vacuum to give 6.7 gm of pure ranolazine (HPLC Purity: 99.97%).

Content of Impurities:

Before Purification: ‘0.58 RRt’ impurity: 0.01%; ‘1.16 RRt’ impurity: 0.03%; dimer impurity-1: 0.05%; dimer impurity-2: 0.02%; dimer impurity-3: 0.02%; impurity-4: 0.07%; impurity-5: 0.01%; and impurity-6: 0.17%.

After Purification: ‘0.58 RRt’ impurity: Below detection limit; ‘1.16 RRt’ impurity: 0.02%; dimer impurity-1: Below detection limit; dimer impurity-2: Below detection limit; dimer impurity-3: Below detection limit; impurity-4: Below detection limit; impurity-5: Below detection limit; and impurity-6: Below detection limit.

### Example 14

Purification of Crude Ranolazine

Crude ranolazine (10 gm, HPLC Purity: 99.71%) was added to dimethylsulfoxide (20 ml) and then heated at 50-55° C. to form a clear solution. The solution was stirred for 10 minutes at 50-55° C. followed by the addition of isopropanol (100 ml) over a period of 30 minutes at 50-55° C., and then stirred for 10 minutes at the same temperature. The resulting solution was cooled to 25-35° C. and stirred for 8-10 hours. The resulting solid was filtered, the product was washed with isopropanol (10 ml) followed by suction drying and then drying at 50-55° C. under vacuum to give 7.4 gm of pure ranolazine (HPLC Purity: 99.87%).

Before Purification: ‘1.16 RRt’ impurity: 0.07%; dimer impurity-3: 0.17%; and impurity-4: 0.04%.

After Purification: ‘1.16 RRt’ impurity: 0.03%; dimer impurity-3: 0.07%; and impurity-4: 0.01%.

### Example 15

Purification of Crude Ranolazine

Crude ranolazine (1 gm, HPLC Purity: 99.71%) was taken in dimethylsulfoxide (2 ml) and then heated at 50-55° C. to form a clear solution. The solution was stirred for 10 minutes at 50-55° C. followed by the addition of ethyl acetate (10 ml) over a period of 30 minutes at 50-55° C. The resulting solution was cooled to 25-35° C. and stirred for 6-8 hours. The resulting mass was then allowed to cool at 0-5° C. and stirred for 4 hours at the same temperature. The resulting solid was filtered, the product was washed with ethyl acetate (1 ml) followed by suction drying and then drying at 50-55° C. under vacuum to give 0.66 gm of pure ranolazine (HPLC Purity: 99.81%).

Before Purification: ‘1.16 RRt’ impurity: 0.07%; dimer impurity-3: 0.17%; and impurity-4: 0.04%.

After Purification: ‘1.16 RRt’ impurity: 0.05%; dimer impurity-3: 0.08%; and impurity-4: Below detection limit.

### Example 16

Purification of Crude Ranolazine

Crude ranolazine (1 gm, HPLC Purity: 99.71%) was added to dimethylsulfoxide (2 ml) and then heated at 50-55° C. to form a clear solution. The solution was stirred for 10 minutes at 50-55° C. followed by the addition of acetone (10 ml) over a period of 30 minutes at 50-55° C. The resulting solution was cooled to 25-35° C. and stirred for 6-8 hours. The resulting mass was then allowed to cool at 0-5° C. and stirred for 4 hours at the same temperature. The resulting solid was filtered, the product was washed with pre cooled acetone (1 ml) followed by suction drying the material and then drying at 50-55° C. under vacuum to give 0.54 gm of pure ranolazine (HPLC Purity: 99.89%).

Content of Impurities:

Before Purification: ‘1.16 RRt’ impurity: 0.07%; dimer impurity-3: 0.17%; and impurity-4: 0.04%.

After Purification: ‘1.16 RRt’ impurity: 0.06%; dimer impurity-3: 0.04%; and impurity-4: Below detection limit.

### Example 17

Purification of Crude Ranolazine

Crude ranolazine (1 gm, HPLC Purity: 99.71%) was added to dimethylsulfoxide (2 ml) and then heated at 50-55° C. to form a clear solution. The solution was stirred for 10 minutes at 50-55° C. followed by the addition of water (10 ml) over a period of 30 minutes at 50-55° C. The resulting solution was cooled to 25-35° C. and stirred for 8-10 hours. The resulting solid was filtered, the product was washed with water (1 ml) followed by suction drying and then drying at 50-55° C. under vacuum to give 0.68 gm of pure ranolazine (HPLC Purity: 99.87%).

Content of Impurities:

Before Purification: ‘1.16 RRt’ impurity: 0.05%; dimer impurity-3: 0.17%; and impurity-4: 0.04%.

After Purification: ‘1.16 RRt’ impurity: 0.04%; dimer impurity-3: 0.07%; and impurity-4: 0.01%.

### Example 18

Purification of Crude Ranolazine

Crude ranolazine (1 gm, HPLC Purity: 99.71%) was added to N,N-dimethylacetamide (2 ml) and then heated at 50-55° C. to form a clear solution. The solution was stirred for 10 minutes at 50-55° C. followed by the addition of isopropanol (10 ml) over a period of 30 minutes at 50-55° C., and then stirred for 10 minutes at the same temperature. The resulting solution was cooled to 25-35° C. and stirred for 6-8 hours. The resulting mass was then allowed to cool at 0-5° C. and stirred for 4 hours at the same temperature. The resulting solid was filtered, the product was washed with isopropanol (1 ml) followed by suction drying and then drying at 50-55° C. under vacuum to give 0.78 gm of pure ranolazine (HPLC Purity: 99.79%).

Content of Impurities:

Before Purification: ‘1.16 RRt’ impurity: 0.05%; dimer impurity-3: 0.17%; and impurity-4: 0.04%.

After Purification: ‘1.16 RRt’ impurity: 0.04%; dimer impurity-3: 0.08%; and impurity-4: 0.01%.

### Example 19

Purification of Crude Ranolazine

Crude ranolazine (1 gm, HPLC Purity: 99.71%) was added to N,N-dimethylacetamide (2 ml) and then heated at 50-55° C. to form a clear solution. The solution was stirred for 10 minutes at 50-55° C. followed by the addition of ethyl acetate (10 ml) over a period of 30 minutes at 50-55° C., and then stirred for 10 minutes at the same temperature. The resulting solution was cooled to 25-35° C. and stirred for 6-8 hours. The resulting mass was then allowed to cool at 0-5° C. and stirred for 4 hours at the same temperature. The resulting solid was filtered, the product was washed with ethyl acetate (1 ml) followed by suction drying and then drying at 50-55° C. under vacuum to give 0.65 gm of pure ranolazine (HPLC Purity: 99.72%).

Content of Impurities:

Before Purification: ‘1.16 RRt’ impurity: 0.05%; dimer impurity-3: 0.17%; and impurity-4: 0.04%.

After Purification: ‘1.16 RRt’ impurity: 0.03%; dimer impurity-3: 0.07%; and impurity-4: Below detection limit.

### Example 20

Purification of Crude Ranolazine

Crude ranolazine (1 gm, HPLC Purity: 99.71%) was added to N,N-dimethylacetamide (2 ml) and then heated at 50-55° C. to form a clear solution. The solution was stirred for 10 minutes at 50-55° C., followed by the addition of water (10 ml) over a period of 30 minutes at 50-55° C., and then stirred for 10 minutes at the same temperature. The resulting solution was cooled to 25-35° C. and stirred for 6-8 hours. The resulting mass was then allowed to cool at 0-5° C. and stirred for 4 hours at the same temperature. The resulting solid was filtered, the product was washed with water (1 ml) followed by suction drying and then drying at 50-55° C. under vacuum to give 0.70 gm of pure ranolazine (HPLC Purity: 99.85%).

Content of Impurities:

Before Purification: ‘1.16 RRt’ impurity: 0.05%; dimer impurity-3: 0.17%; and impurity-4: 0.04%.

After Purification: ‘1.16 RRt’ impurity: 0.05%; dimer impurity-3: 0.05%; and impurity-4: 0.01%.

### Example 21

Purification of Crude Ranolazine

Crude ranolazine (10 gm, HPLC Purity: 99.42%) was added to a mixture of N,N-dimethylacetamide (20 ml) and acetone (100 ml), the resulting mixture was heated at 50-55° C. to form a clear solution, and then stirred for 10 minutes at 50-55° C. The resulting solution was cooled to 25-35° C. and stirred for 6-8 hours. The resulting solid was filtered, the product was washed with pre cooled acetone (10 ml) followed by suction drying and then drying at 50-55° C. under vacuum to give 6.7 gm of pure ranolazine (HPLC Purity: 99.92%).

Content of Impurities:

Before Purification: ‘0.58 RRt’ impurity: 0.01%; ‘1.16 RRt’ impurity: 0.03%; dimer impurity-1: 0.05%; dimer impurity-2: 0.02%; dimer impurity-3: 0.02%; impurity-4: 0.07%; impurity-5: 0.01%; and impurity-6: 0.17%.

After Purification: ‘0.58 RRt’ impurity: Below detection limit; ‘1.16 RRt’ impurity: 0.03%; dimer impurity-1: Below detection limit; dimer impurity-2: Below detection limit; dimer impurity-3: Below detection limit; impurity-4: Below detection limit; impurity-5: Below detection limit; and impurity-6: 0.03%.

### Example 22

Purification of Crude Ranolazine

Crude ranolazine (1 gm) was added to N,N-dimethylformamide (2 ml) and then heated at 50-55° C. to form a clear solution. The solution was stirred for 10 minutes at 50-55° C. followed by the addition of isopropanol (10 ml) over a period of 30 minutes at 50-55° C., and then stirred for 10 minutes at the same temperature. The resulting solution was cooled to 25-35° C. and stirred for 8-10 hours. The resulting solid was filtered, the product was washed with isopropanol (10 ml) followed by suction drying and then drying under vacuum at 50-55° C. to give 0.65 gm of pure ranolazine.

### Example 23

### Preparation of 1-[4-[2-Hydroxy-3-(2-methoxy-phenoxy)-propyl]-piperazin-1-yl]-3-(2-methoxy-phenoxy)-propan-2-ol (Dimer Compound or Dimer Impurity-3)

3-(2-Methoxyphenoxy)-1-piperazinyl-propane-2-ol (15 gm) and 2-[(2-methoxyphenoxy)methyl]oxirane (10.2 gm) were added to methanol (150 ml) at 25-30° C., the resulting mixture was heated to reflux (64-65° C.), and then stirred for 4 hours at reflux temperature. The reaction mass was cooled to 25-30° C. over a period of 2 to 3 hours, and followed by stirring for 2 hours. The resulting solid was filtered, washed with methanol (30 ml), suction dried and followed by drying under vacuum at 50-60° C. for 6-8 hours to give 10 gm of 1-[4-[2-hydroxy-3-(2-methoxy-phenoxy)-propyl]-piperazin-1-yl]-3-(2-methoxy-phenoxy)-propan-2-ol.

Unless otherwise indicated, the following definitions are set forth to illustrate and define the meaning and scope of the various terms used to describe the invention herein.

The term “ranolazine” as used herein refers to a racemic mixture of enantiomeric forms of ranolazine or an enatiomerically enriched form of ranolazine.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude ranolazine or a pharmaceutically acceptable salt thereof” as used herein refers to ranolazine or a pharmaceutically acceptable salt thereof containing greater than about 0.15 area-%, more specifically greater than about 0.2 area-%, still more specifically greater than about 0.4 area-% and most specifically greater than about 1 area-% of at least one, or more, of the dimer impurity-1, dimer impurity-2, dimer impurity-3, impurity-4, impurity-5, impurity-6, ‘0.58 RRt’, ‘0.78 RRt’, ‘1.02 RRt’, ‘1.07 RRt’, ‘1.16 RRt’ and ‘1.23 RRt’ impurities.

As used herein, the term, “detectable” refers to a measurable quantity measured using an HPLC method having a detection limit of 0.01 area-%.

As used herein, in connection with amount of impurities in ranolazine or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated dimer impurity of ranolazine, ranolazine dimer impurity-3, 1-[4-[2-hydroxy-3-(2-methoxy-phenoxy)-propyl]-piperazin-1-yl]-3-(2-methoxy-phenoxy)-propan-2-ol, of formula II:

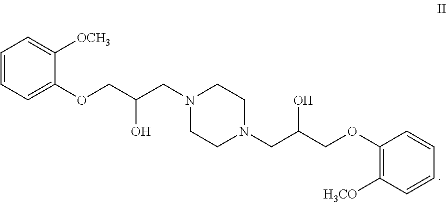

2. Ranolazine or a pharmaceutically acceptable salt thereof comprising the 1-[4-[2-hydroxy-3-(2-methoxy-phenoxy)-propyl]-piperazin-1-yl]-3-(2-methoxy-phenoxy)-propan-2-ol impurity (ranolazine dimer impurity-3) of claim 1 in an amount of less than 0.15 area-% as measured by HPLC, wherein the ranolazine has a purity of about 99% to about 99.99% as measured by HPLC.

3. (canceled)

4. Ranolazine of claim 2, comprising the ranolazine dimer impurity-3 in an amount of about 0.01 area-% to about 0.1 area-%; and wherein the ranolazine further comprises at least one, or more, of a ‘0.58 RRt’ impurity, a ‘0.78 RRt’ impurity, a ‘1.02 RRt’ impurity, a ‘1.07 RRt’ impurity, a ‘1.16 RRt’ impurity, a ‘1.23 RRt’ impurity, a dimer impurity-1, a dimer impurity-2, an impurity-4, an impurity-5 and an impurity-6, each, in an amount of less than 0.15 area-% as measured by HPLC; wherein the dimer impurity-1 is 1,3-bis-(2-methoxyphenoxy)-propan-2-ol, the dimer impurity-2 is N-(2,6-dimethyl-phenyl)-2-[4-[(2,6-dimethyl-phenylcarbamoyl)-methyl]-piperazin-1-yl]-acetamide, the impurity-4 is 2-[(2-methoxyphenoxy)methyl]oxirane, the impurity-5 is 2-chloro-N-(2,6-dimethylphenyl)acetamide, and the impurity-6 is 4-[(2,6-dimethylphenyl)aminocarbonylmethyl]piperazine.

5. Ranolazine of claim 2, having a non-detectable amount of the ranolazine dimer impurity-3 as measured by HPLC; and wherein the pharmaceutically acceptable salt of ranolazine is a dihydrochloride, a dihydrobromide, an oxalate, a maleate, a fumarate, a besylate, a tosylate or a tartrate salt.

6. (canceled)

7. (canceled)

8. Ranolazine of claim 4, having a non-detectable amount of one, or more, of the ‘0.58 RRt’, ‘0.78 RRt’, ‘1.02 RRt’, ‘1.07 RRt’, ‘1.16 RRt’, ‘1.23 RRt’ impurities, dimer impurity-1, dimer impurity-2, impurity-4, impurity-5 and impurity-6 as measured by HPLC.

9. (canceled)

10. (canceled)

11. A process for preparing the highly pure ranolazine or a pharmaceutically acceptable salt thereof of claim 2, comprising:
a) dissolving or extracting crude ranolazine in a first solvent or a solvent medium comprising the first solvent and a second solvent to produce a first solution, wherein the first solvent is selected from the group consisting of a polar aprotic solvent, a ketone, and mixtures thereof, and wherein the second solvent is selected from the group consisting of water, an alcohol, an ester, and mixtures thereof;
b) optionally, subjecting the first solution to carbon treatment or silica gel treatment;
c) optionally, combining the first solution obtained in step-(a) or step-(b) with the second solvent to produce a second solution or suspension;
d) isolating and/or recovering highly pure ranolazine substantially free of the impurities either from the first solution obtained in step-(a) or step-(b), or from the second solution or suspension obtained in step-(c); and
e) optionally, converting the highly pure ranolazine obtained in step-(d) into a pharmaceutically acceptable salt thereof.

12. The process of claim 11, wherein the first solvent used in step-(a) is selected from the group consisting of acetone, methyl ethyl ketone, methyl isobutyl ketone, methyl tert-butyl ketone, N,N-dimethylformamide, N,N-dimethylacetamide, dimethylsulfoxide, tetrahydrofuran, and mixtures thereof; and wherein the second solvent used in step-(a) or step-(c) is selected from the group consisting of water, methanol, ethanol, n-propanol, isopropyl alcohol, isobutanol, n-butanol, tert-butanol, amyl alcohol, isoamyl alcohol, ethyl acetate, methyl acetate, isopropyl acetate, tert-butyl methyl acetate, ethyl formate, and mixtures thereof.

13. The process of claim 12, wherein the first solvent is selected from the group consisting of N,N-dimethylacetamide, dimethylsulfoxide, tetrahydrofuran, and mixtures thereof; and wherein the second solvent is selected from the group consisting of water, methanol, ethanol, isopropyl alcohol, ethyl acetate, and mixtures thereof.

14. The process of claim 11, wherein the first solution in step-(a) is prepared by dissolving or extracting the crude ranolazine free base in the first solvent or in the solvent medium comprising the first and second solvents at a temperature of above about 25° C.; wherein the first solution obtained in step-(a) is optionally stirred at a temperature of about 25° C. to about 110° C. for about 15 minutes to about 8 hours; wherein the combining in step-(c) is accomplished by adding the first solution to the second solvent or by adding the second solvent to the first solution at a temperature of about 25° C. to about 110° C.; wherein the isolation of highly pure ranolazine base in step-(d) is carried out by cooling, seeding, partial removal of the solvent from the solution, by combining an anti-solvent with the solution, or a combination thereof; wherein the recovering in step-(d) is carried out by filtration, filtration under vacuum, decantation, centrifugation, filtration employing a filtration media of a silica gel or celite, or a combination thereof; and wherein the pure ranolazine or a pharmaceutically acceptable salt thereof obtained is further dried under vacuum or at atmospheric pressure, at a temperature of about 35° C. to about 70° C.

15. The process of claim 14, wherein the ranolazine free base is dissolved in the solvent or solvent medium at a temperature of about 25° C. to about 110° C.; wherein the reaction mass obtained after completion of the addition process in step-(c) is stirred at a temperature of about 25° C. to about 110° C. for about 10 minutes to about 10 hours; and wherein the isolation in step-(c) is carried out by cooling the solution while stirring at a temperature of about 0° C. to about 30° C. for about 30 minutes to about 20 hours.

16. (canceled)

17. (canceled)

18. (canceled)

19. (canceled)

20. (canceled)

21. (canceled)

22. (canceled)

23. (canceled)

24. (canceled)

25. The process of claim 11, wherein the ranolazine or a pharmaceutically acceptable salt thereof obtained has less than about 300 parts per million (ppm) methanol, less than about 1000 ppm acetone, less than about 50 ppm methylene chloride, less than about 500 ppm methyl ethyl ketone, less than about 500 ppm ethyl acetate, less than about 300 ppm cyclohexane, and less than about 7000 ppm N,N-dimethylacetamide; and wherein the ranolazine or a pharmaceutically acceptable salt thereof obtained has the overall level of organic volatile impurities in an amount of less than about 1500 parts per million.

26. (canceled)

27. The highly pure ranolazine or a pharmaceutically acceptable salt thereof of claim 2, further comprising one or more pharmaceutically acceptable excipients to form a pharmaceutical composition.

28. (canceled)

29. (canceled)

30. (canceled)

31. (canceled)

32. (canceled)

33. (canceled)

34. The pharmaceutical composition of claim 27, wherein the pharmaceutical composition is a solid dosage form, an oral suspension, a liquid, a powder, an elixir, an aerosol, syrups or an injectable solution.

35. The pharmaceutical composition of claim 27, wherein the highly pure ranolazine or a pharmaceutically acceptable salt thereof has a D₉₀ particle size of less than or equal to about 400 microns.

36. The pharmaceutical composition of claim 35, wherein the D₉₀ particle size is less than or equal to about 60 microns.

37. (canceled)
